(19) United States
(12) Patent Application Publication
(10) Pub. No.: US 2010/0316734 A1
(43) Pub. Date: Dec. 16, 2010

(54) METHODS OF THERMOGRAPH-GUIDED MEDICAL TREATMENT

(75) Inventors: Julie Hart, Brooks, CA (US); Christopher Cozzie, Cardiff, CA (US)

Correspondence Address: PROCOPIO, CORY, HARGREAVES & SAVITCH LLP, 525 B STREET, SUITE 2200, SAN DIEGO, CA, 92101, US

(73) Assignee: Christopher Cozzie, Cardiff, CA (US)

(21) Appl. No.: 12/862,663

(22) Filed: Aug. 24, 2010

Related U.S. Application Data
(63) Continuation of application No. 12/144,571, filed on Jun. 23, 2008, now abandoned.
(60) Provisional application No. 60/945,877, filed on Jun. 22, 2007.

(51) Int. Cl. A61K 33/26; A61K 33/24; A61K 33/22; A61P 29/00; A61P 39/06; A61P 35/00

(52) U.S. Cl. 424/646; 424/655; 424/657

(57) ABSTRACT

Methods and systems for treatment based on thermographic images of a patient to detect temperature patterns indicative of an evolving disease process. Guided by the thermographic image obtained, a treatment means can be positioned on the skin of the imaged body part. The treatment means comprises a medicament that reduces risk factor(s) associated with the progression of the evolving disease process.

### PRIORITY CLAIM AND RELATED APPLICATION

This application claims the priority as a continuation under 35 U.S.C. 120 to U.S. patent application Ser. No. 12/144,571, filed Jun. 23, 2008, and entitled “Methods of Thermograph-Guided Medical Treatment,” which in turn claims the benefit of U.S. Provisional Application No. 60/945,877, filed Jun. 22, 2007 and entitled “Methods of Thermograph-Guided Medical Treatment,” both of which are incorporated by reference as part of the specification of this application.

### BACKGROUND

Current medical practice in the successful treatment of cancer is early diagnosis of tumors. Mammography has been used as a screening tool for the early detection of breast cancer tumors since its approval by the FDA in 1982. Mammography can show changes in the breast up to two years before a patient or physician can feel them.

Early detection of breast tumors by mammography can only identify a tumor that has already formed. Because mammography is limited to the diagnosis of pre-existing tumors, most women will go on to require surgery, radiation and/or chemotherapy in order to treat the tumor detected by mammography.

### SUMMARY

Thus, there is a growing need for diagnosing the potential for disease, such as breast cancer, thereby providing an opportunity for preventative measures to be taken. There is also a need for improved preventative treatments once the increased risk for the development of disease is identified. There is also a need for improved diagnostic procedures for monitoring breast health that is safer, more comfortable to the patient and more reliable in its imagery of breast tissue from all women regardless of age or hormonal state.

Methods are disclosed herein to address the above-described shortcomings of mammography as a tool for disease risk assessment and monitoring.

In one embodiment, disclosed is a method of treatment of obtaining a thermographic image of a body part of a patient, the thermographic image detects temperature patterns indicative of an evolving disease process; and positioning a treatment device on a region of skin of the imaged body part according to the thermographic image obtained, the treatment device includes a medicament, the medicament reduces a risk factor associated with the progression of the evolving disease process.

In another embodiment, disclosed is a method of treating an evolving disease process including obtaining a thermographic image of a body part of a patient, the thermographic image detecting temperature patterns indicative of an evolving disease process; analyzing the thermographic image to identify temperature patterns indicative of an evolving disease process; locating, according to the thermographic image obtained, a region of the body part identified as having temperature patterns indicative of an evolving disease process, the region of the body part is covered by skin, and positioning a treatment device on the region of skin of the body part, the treatment device including a medicament.

Other features and advantages will be apparent from the following description of various embodiments, which illustrate, by way of example, the principles of the disclosed devices and methods.

### BRIEF DESCRIPTION OF THE DRAWINGS

FIG. 1 shows a thermograph of pre-cancerous tissue compared to normal tissue.

FIG. 2 shows a flowchart illustrating an exemplary method of treatment.

### DETAILED DESCRIPTION

Digital infrared thermal imaging or thermography permits high resolution passive remote thermal radiation sensing to non-invasively assess the temperature of the interior of a body as well as the presence of inflammation in tissues. Thermograms produced by infrared camera equipment and other surface temperature measurements detect abnormal temperature patterns indicative of the presence of an evolving disease process such as those that result from an inflammatory process. Information on this process can be found in for example, Hirschenbein N. “Preventing Breast Cancer” Healthy Aging, p 31-34, November/December 2006, which is incorporated herein by reference as part of this specification.

There are multiple lines of compelling evidence supporting the association between inflammation and cancer. Further, epidemiologic and clinical research indicates an increased risk of certain cancers in the setting of chronic inflammation. Many of the same processes involved in inflammation (e.g., leukocyte migration, dilatation of local vasculature with increased permeability and blood flow, angiogenesis) also contribute to tumor development. The ability of thermography to detect as heat the metabolic and physiologic changes involved in the initiation of a tumor provide the clinician and the patient an opportunity to intervene in the early stages of an evolving disease before a tumor develops. The patient can take appropriate preventative measures in order to avoid future development of a full-blown disease.

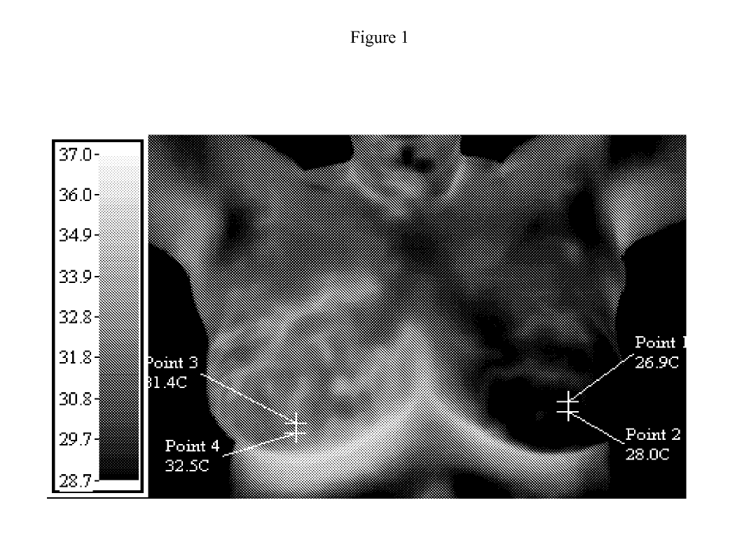

FIG. 1 shows an exemplary thermograph taken by digital infrared imaging. The patient's right breast shows increased chemical and blood vessel activity detected as heat in a thermograph as shown by regions of white or gray compared to the dark regions of normal breast tissue (left breast). Increased metabolic activity and increased vascular circulation (due to angiogenesis) in pre-cancerous tissue as well as any areas surrounding a developing cancer are detectable by the thermography but cannot be detected by mammography.

### Detoxification Through the Skin

One exemplary preventative measure includes detoxification through the skin using a dermal patch (described in more detail below). The dermal patch can be used to reduce inflammatory, glycation and oxidative stress factors within the pre-cancerous tissue. The patch can also be used to increase the antioxidant reserves, tissue repair capabilities and acid-base balance in the breast tissue of a woman identified via thermography as having pre-cancerous tissue. Reducing risk factors that promote an evolving disease process or pre-cancerous condition into an active cancer can prevent or delay the development of the disease.

Detoxification patches act like a poultice to remove toxins from the body through the skin. A poultice can be made of a porous material. A solvent of the poultice equilibrates with target solute in a body and by passive diffusion solute enters the poultice through the skin thereby having “detoxifying” effects. After an adequate time passes for this process to occur, the poultice is removed and with it the dissolved solutes or toxins.

The patch can be, for example, a pouch or other sealed enclosure or bag formed of a permeable fabric such as gauze, muslin, linen, or white cotton sheeting. The size and shape of the patch can vary. The ingredients that permeate the patch can provide treatment that reduces risk factors associated with the progression of the evolving disease process. For example, the ingredients in the patch can provide anti-inflammatory and anti-oxidant properties. Similarly, ingredients in the patch can receive or extract toxins from the body such as heavy metals, free radicals and chemicals from the body. Thus, the mesh-like or porous patch can act in a uni-directional or bi-directional manner.

The ingredients of the patch can include, but are not limited to one or a combination of: a mineral, a silicon-based mineral, a far infrared emitting element, clay, tourmaline, citrine, wood vinegar, bamboo vinegar, vitamin C, dokudami, loquat leaf, amygdalin, vitamin B17, laetrile, chitosan, chitin, turmeric, curcumen, milk thistle, pau d'arco. The patch may include powdered tourmaline crystal, bamboo vinegar, vitamin C, dokudami, loquat leaf, and chitosan. An exemplary mixing ratio of the above ingredients can be as follows (% weight): tourmaline crystal 30%, bamboo vinegar 21%, wood vinegar 20%, chitosan 1.5%, loquat leaf 1.5%, dokudami 7%, Vitamin C 1.5%, vegetable fiber 7.5% and dextrin 10%. The ingredients of the patch can vary according to the application of the obtained composition. For example, additional active ingredients such as curcumen, milk thistle and/or pau d'arco can be added to the above ratio of ingredients.

Silicon-based minerals such as granite, perlite, pitchstone, and tourmaline can be used as main components. These minerals radiate electromagnetic waves (feeble energy) and release anions. The mineral in the patch can be tourmaline. Tourmaline is both pyroelectric and piezoelectric. If a specimen is put under pressure, or a temperature change, it will generate an electrical charge. Tourmaline is best known as one of the only minerals to emit far infrared heat and negative ions. Pyroelectricity of tourmaline results in adsorbing properties such as fixing heavy metal ions and adsorbing malodorous composition particles. Tourmaline can be milled into a powder using No. 40 mesh. For example, the particle size of the powdered tourmaline crystal using 40 mesh can be the size of 420 μm. The mineral in the patch can be a multi-elemental mineral, such as a mixture of tourmaline and citrine. The mineral powders can be used without further processing. Alternatively, the powders can also be used after they are mixed with water, whether heated or pressurized, so that the clear liquid part of the water dries into a powder by vacuum-freeze drying or by spray drying methods.

Bamboo vinegar is a material analogous to pyroligneous acid. It represents the upper part of the liquid obtained by cooling the gas generated in a process of dry distillation of bamboo or in a process of producing bamboo charcoal, as in pyroligneous acid (i.e., wood vinegar). It contains acetic acid and methyl alcohol. The substance has sterilization, deodorization and humidity conditioning effects due to its excellent adsorbability.

Other ingredients can be selected based upon their anti-inflammatory, anti-oxidant and anti-cancer properties. For example, Vitamin C (ascorbic acid) can be obtained from citrus extracts such as grapefruit extract or orange extract. Vitamin C and citrus extracts provide anti-oxidant properties and anti-mutagen properties. The scent of the citrus extracts provides a pleasant aromatherapy. Dokudami (houttuynia herb) is a plant known to have strong adsorption properties. Loquat leaf (Eriobotrya japonica Lindley) contains malic acid, tartaric acid, citric acid tannate, carotene, vitamins A, Band C. Its leaves are mainly used for their anti-inflammatory effect. They also contain amygdalin (vitamin B17), which is also known as the anti-cancer vitamin. The rhizome (root) of turmeric (Curcuma longa Linn or curcumin) has anti-inflammatory, antioxidant, and anti-cancer properties. Milk thistle has been shown to provide anti-inflammatory properties and is also known for its beneficial effects on general breast health in females. Pau d'arco has been used in South America as a cancer treatment for several decades. It is also known as taheebo, ipe roxo and cancer tea. Chitosan can be obtained from chitinous substance included in carapaces of conchostracan such as prawn and crab. Chitosan products have been used by water companies to trap toxins, grease, heavy metals and oils. Chitosan is also used in the medical profession to promote wound healing of burns and skin inflammation.

### Reduction of Inflammation in Thermo-Imaged Pre-Cancerous Breast Tissue

As discussed above, thermograms detect abnormal temperature patterns indicative of the presence of inflammation and an evolving disease process. Studies have shown a relationship between microvessel density and thermographic hot areas surrounding breast tumors. Information on this process can be found in, for example, Yahara et al. Surg Today 33(4):243-8 (2003), which is incorporated herein by reference as part of this specification. As shown in FIG. 1, the chemical and blood vessel activity in pre-cancerous tissue (right breast) and the surrounding areas of the developing breast cancer is higher than in the normal breast tissue (left breast) and is detected as heat or hot spots in a thermograph. The physiologic information provided by thermograms allow for the detection of pre-cancerous areas of the breast tissue. In turn, the opportunity is provided to take appropriate preventative steps in order to avoid the development of full-blown disease by reducing certain risk factors associated with the progression of the evolving disease process.

It has been suggested that one of the first biochemical signals of change in breast cells may be expressed as an inflammatory response. Further, a theoretical model of the inflammatory process has been suggested and predictive linkages shown among stimuli in the breast microenvironment and the development of breast cancer. Information on this process can be found in, for example, Lithgow et al., Biol Res Nurs. 7(2):118-29 (2005), which is incorporated herein by reference as part of this specification. We provide here a method of anti-inflammatory therapy by a dermal detoxification patch to prevent or delay progression of precancerous tissue identified by thermographic imaging into diseased tissue.

One exemplary method of treatment includes performing thermography, for example, using an IRIS infrared imaging system, to identify regions of pre-cancerous tissue in the breast of a patient. The advanced computerized infrared camera systems detect heat patterns in the breast. Chemical and blood vessel activity in pre-cancerous tissue and the area surrounding a developing breast cancer is higher than normal breast tissue. Increased blood supply can cause abnormal heat patterns.

The room temperature can be carefully maintained and monitored due to the key role temperature plays in the images obtained. Patients also can equilibrate to their resting temperatures prior to imaging. The imaging can be a two part session in which a baseline reading is taken followed by a functional or stress reading. The baseline reading of frontal and bilateral oblique images can be taken after the patient has rested and equilibrated to the ambient temperature of the room. The functional reading can be taken to register a patient's response to an autonomic challenge of sympathetic vasoconstriction and/or vasodilation. The challenge can be a simple warm water soak of the patient's hand. This allows technicians to observe the presence of non-responsive blood vessels. Such non-responsive blood vessels can be indicative of a malignant neoplasm. The high-resolution frontal and bilateral oblique diagnostic images obtained at baseline and again after the autonomic challenge can then be analyzed according to methods known in the art to identify and locate areas of abnormal breast tissue. Thermograms are interpreted and scored for different abnormalities.

A patient with an abnormal thermogram can then be placed on a comprehensive evaluation of risk factors and an appropriate treatment protocol initiated. In an exemplary treatment protocol, one or more detoxification patches are positioned on the skin covering regions of the breast identified by thermography as expressing or having abnormal temperature patterns or temperature patterns indicative of an evolving disease process. The positioning of the patch is assisted by or is performed according to the thermographic map of pre-cancerous regions or hot spots provided by the infrared imaging system. The patches are placed into direct contact with the targeted portion of the skin where care or treatment thereof is desired. For example, the regions of skin directly overlying tissue expressing the abnormal temperature pattern can be located according to the thermographic image and the treatment device positioned on that region or those regions of skin. The patch(es) can be fixed in place by an adhesive bandage.

Treatment protocols can vary depending on the size and severity of pre-diseased regions identified. It should be noted that use of the patches is also not limited to only the breast, but can be applied to any body part. For example, additional patches can be positioned according to reflexology points on the patient's foot. Treatment protocols can vary also with respect to number of days and the length of time per day the patch is positioned on the patient's skin. Patches can be used daily between, for example, around 5 to around 15 hours per day. The course of treatment can be, for example, between around 5 days up to around 90 days. One exemplary treatment protocol includes treatments between around 7 to around 10 hours nightly while the patient is sleeping, for a minimum of 5 days. Patches are removed each morning and can be saved for further analysis (described in more detail below). The detoxification patch can be part of a kit. The kit can include at least one patch enclosed in a protective wrapper, at least one adhesive sheet covered by backing paper for adhering the patch to the patient's skin. Each kit can contain the appropriate number of patches and adhesive sheets needed for a course of therapy (i.e. number of days of therapy would be equal to the number of patches in the kit).

Following the treatment protocol, patients can undergo follow-up thermographic imaging. The images obtained can provide evidence of reduction in inflammation or “hot spots” due to treatment with the patches. Further thermographic images can be obtained to monitor lasting efficacy of treatments and, if applicable, additional courses of treatments. In addition, correlative lab values known to identify inflammatory processes can be performed on patients prior to and following treatment with the detoxification patches.

The used detoxification patches can be analyzed to assess efficacy of treatment. For example, presence of factors related to inflammation found in the used detoxification patches can be analyzed by methods known in the art. Similarly, removal of other factors, such as toxins and heavy metals, can be analyzed by known methods. For example, levels of benzene, nickel, isopropyl alcohol, thallium, methyl alcohol, thulium, aluminum, arsenic, cadmium, asbestos, copper, azo dyes, lead, PCBs, and mercury collected in the patch after use can be analyzed by methods known in the art. Similarly, hair samples taken prior to and after treatment can be analyzed to show reduction in selected toxins and heavy metals according to methods known in the art. Dark field microscopy can also be used to identify other items extracted from the tissues. The analysis of used detoxification patches can help to determine what the next step of treatment can be or what other preventative measures can be taken. The presence of factors identified on the used patches could also indicate the early stages of other types of disease processes not yet identified.

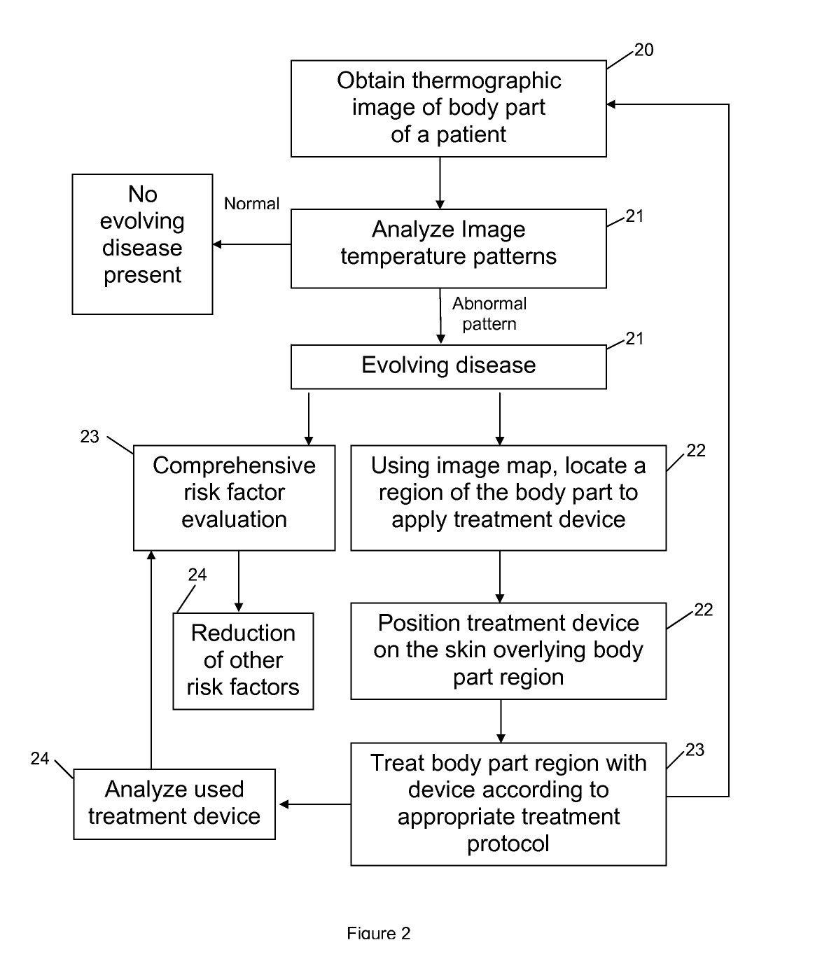

FIG. 2 is a flowchart illustrating one exemplary method of treatment. In this method of treatment a thermographic image of a body part of a patient is obtained (box 205) temperature patterns of the thermographic image(s) are analyzed (box 210) by techniques known in the art. If abnormal temperature patterns are found this is indicative of an evolving disease process (box 215). Once the abnormal thermogram is identified, patients can undergo a two-prong approach of disease prevention.

FIG. 2 shows that one aspect of the two-prong approach can include a detoxification patch treatment protocol. The thermographic image showing abnormal regions of heat emission can be used like a map to locate a region of the body part on which to apply the treatment device or detoxification patch (box 220). Once a region of the body part is identified, the treatment device is positioned on the skin overlying the target region (box 225). The device can be affixed to the skin by methods known in the art, such as an adhesive strip or bandage or the like. The body part region is then treated with the device according to a treatment protocol (box 230) such as those protocols described above. Following treatment with the device, the patient can undergo follow-up thermographic imaging to assess efficacy of treatment (box 205).

FIG. 2 shows that another aspect of the two-prong approach can include placing patients on a comprehensive evaluation of risk factors (box 235). The evaluation can include assessing hormone levels, nutrient and dietary intake levels, intestinal tract health, metabolic health, glycation, inflammation, oxidative stress, antioxidant reserves, tissue repair deficits, acid-base balance, stress and psychological factors, heavy metal toxicities and exposure to pollutants. The used treatment patches also can be analyzed (box 245) according to methods known in the art to help identify and evaluate risk factors of the evolving disease process. Based on the findings of the analysis of used patches as well as the results of the comprehensive risk factor evaluation, supplemental treatment(s) can be performed and/or lifestyle changes made to reduce the identified risk factors of the evolving disease process (box 240).

It should be noted that use of the detoxification patches is not limited to treatment of inflammation and pre-cancerous tissue. Information on the 300 chemical pollutants in breastmilk can be found in, for example, http://www.foeeurope.org/publications/2006/toxic_inheritance.pdf, June 2006, which is incorporated herein by reference as part of this specification. Thus, these detoxification patches can be used to remove long-lived toxins, lipophilic chemicals and heavy metals from breastmilk of lactating women. Similarly, the detoxification patches can be used in women who are pregnant or plan to become pregnant to prevent transmission of these toxins through the placenta to the fetus.

While the present methods are described as being applied to the human body, it is to be understood that they may also be applied to the body of any animal and are applicable to veterinary uses.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Only a few examples and implementations are disclosed. Variations, modifications and enhancements to the described examples and implementations and other implementations may be made based on what is disclosed.

What is claimed is:

1. A method of treatment comprising:
obtaining a thermographic image of a body part of a patient, wherein the thermographic image detects temperature patterns indicative of an evolving disease process; and
positioning a treatment device on a region of skin of the imaged body part according to the thermographic image obtained, the treatment device comprising a medicament, wherein the medicament reduces a risk factor associated with the progression of the evolving disease process.

2. The method of claim 1, wherein the medicament comprises an anti-inflammatory agent.

3. The method of claim 2, wherein the medicament further comprises an antioxidant agent.

4. The method of claim 1, wherein the medicament comprises a mineral.

5. The method of claim 4, wherein the mineral is tourmaline.

6. The method of claim 4, wherein the medicament further comprises a wood vinegar extract.

7. The method of claim 6, wherein the wood vinegar extract is bamboo vinegar extract.

8. The method of claim 1, wherein the medicament comprises tourmaline, bamboo vinegar extract, chitosan, loquat leaf, dokudami and vitamin C.

9. The method of claim 8, wherein the medicament further comprises at least one member of the group consisting of curcumen, milk thistle and pau d'arco.

10. The method of claim 1, wherein the evolving disease process is cancer.

11. The method of claim 10, wherein the body part is a breast and the cancer is breast cancer.

12. The method of claim 1, wherein the risk factor comprises increased inflammation.

13. The method of claim 1, wherein the patient is a mammal.

14. The method of claim 13, wherein the patient is a human.

15. A method of treating an evolving disease process comprising:
obtaining a thermographic image of a body part of a patient, wherein the thermographic image detects temperature patterns indicative of an evolving disease process;
analyzing the thermographic image to identify temperature patterns indicative of an evolving disease process;
locating, according to the thermographic image obtained, a region of the body part identified as having temperature patterns indicative of an evolving disease process, wherein the region of the body part is covered by skin; and
positioning a treatment device on the region of skin of the body part, the treatment device comprising a medicament.
